(19) United States
(12) Patent Application Publication
(10) Pub. No.: US 2004/0019336 A1
(43) Pub. Date: Jan. 29, 2004

(54) Eye wipes and eye wipe dispensers

(76) Inventors: Angela Temple, Austin, TX (US); Gary B. Solomon, Dallas, TX (US)

Correspondence Address: FISH & RICHARDSON P.C., 5000 BANK ONE CENTER, 1717 MAIN STREET, DALLAS, TX, 75201, US

(21) Appl. No.: 10/622,409

(22) Filed: Jul. 18, 2003

Related U.S. Application Data
(60) Provisional application No. 60/396,770, filed on Jul. 18, 2002.

(51) Int. Cl. A61M 35/00

(52) U.S. Cl. 604/294

(57) ABSTRACT

Eye wipes small in size and optionally may include a slot or other cavity to enable a finger to be inserted to control the eye wipe and reduce the chance of a fingernail damaging an eye. The eye wipe may be substantially free from chemicals or other eye irritants and optionally include solutions that are “eye-friendly”, such as saline or other solutions as understood in the art. Still yet, the inventive eye wipes may be substantially free from pulp, filaments, or other fibrous material and may be textured or non-textured. In one embodiment, the eye wipe may be utilized for removing makeup and be saturated with makeup removing solution.

### CROSS-REFERENCES TO RELATED APPLICATIONS

This application claims priority to U.S. Provisional Application Serial No. 60/396,770, filed on Jul. 18, 2002, the entire teachings of which are incorporated herein by reference.

### FIELD OF THE INVENTION

The principles of the present invention are directed to eye wipes, and more particularly, but not by limitation, to eye wipes and dispensers for storing and dispensing the eye wipes.

### BACKGROUND OF THE INVENTION

People use conventional eye wipes, such as tissues, to wipe tears, residue, and make-up from around their eyes, especially near the tear duct. The conventional wipes used for wiping eyes are generally designed for other purposes, such as blowing one's nose, and are over-sized relative to the purpose of wiping one's eyes (see, FIG. 14). The conventional eye wipes are also environmentally wasteful for the purpose of wiping one's eyes. Moreover, extraneous material of the conventional wipes may contact and irritate the eyes. Lotions, fragrances, chemicals, etc. infused in certain conventional wipes can also contact the eyes and cause irritation of the eyes.

Because babies are unable to wipe the residue formed on the eyes from tears, caregivers of the babies routinely have to wipe the eyes of the babies to remove the residue. Using conventional wipes to wipe the eyes of the babies is cumbersome due to the size of the conventional wipes. Further, babies' eyes tend to be more sensitive to the problems discussed above than are adults' eyes. Still yet, newborn babies tend to have a condition that causes a blocked tear duct, which leads to caregivers having to clear discharge from the eyes of the baby. In addition, caregivers tend to be cautious with infants and prefer avoiding contact with the eyes of infants with non-sterile items, such as conventional wipes.

While there are certain eye problems associated with babies, there are medical conditions that are shared by people of all ages and other medical conditions that affect older people. For example, both children and adults may have a similar chronic problem, such as dry eye, that causes the eye to over-produce tears in addition to discharge. About 10 million Americans suffer from dry eye syndrome. Most of these cases result from normal aging of the glands in the eye, but dry eye can occur at any age. It is estimated that nearly 75 percent of people over age 65 experience dry eye syndrome. This syndrome occurs in both men and women, although it is most common in women who are pregnant or post-menopausal. About three million Americans have dry eye as a consequence of Sjögren's Syndrome, and 90 percent of these people are women. People suffering from allergies and those wearing contact lenses have greater risk of developing dry eye. Still yet, there are a number of other conditions that affect peoples' eyes, including pink eye, watery eye, and allergies. Each of these eye conditions results in tears or discharge causing discomfort for the person. In essence, conventional wipes are poorly suited to handle these conditions for at least the reasons discussed above.

Beyond the problems associated with wiping eyes discussed above, there are also practical problems associated with using conventional wipes designed for non-eye wipe purposes. For example, a caregiver or user of eye wipes generally desires to travel “light” and carry a minimum number of items having the smallest volume possible. In the case of the conventional wipes being tissues, carrying tissues and other such items may be bulky due to the size of tissue containers.

### BRIEF SUMMARY OF THE INVENTION

To overcome the problems and shortcomings of conventional eye wipes, the principles of the present invention provide for eye wipes that are small in size and optionally include a slot or other cavity to enable a finger to be inserted to control the eye wipe and reduce the chance of a fingernail damaging an eye. The inventive eye wipe may be substantially free from chemicals or other eye irritants and optionally include solutions that are “eye-friendly”, such as saline or other solutions as understood in the art. Still yet, the inventive eye wipes may be substantially free from pulp, filaments, or other fibrous material and may be textured or non-textured.

A dispenser may approximate the size and shape of the eye wipe to store and dispense the eye wipes. Further, the dispenser may be adapted to secure to other objects, such as a baby carriage, bag strap, diaper bag, or other item generally utilized to care for an infant. The dispenser may keep a pre-moistened eye wipe from drying out or include a chamber in which eye-friendly solution is stored and applied to the eye wipe at time of use. The eye wipes may also be individually wrapped in a dry or pre-moistened state.

### BRIEF DESCRIPTION OF THE DRAWINGS

FIG. 1 is an illustration of an eye wipe according to the principles of the present invention;

FIGS. 2A-2C are illustrations of other embodiments of the eye wipe of FIG. 1;

FIGS. 3A-3C are different views of yet another embodiment of the eye wipe of FIG. 1;

FIGS. 4A-4B illustrate eye wipes of FIG. 2 that are coupled together to be maintained in a dispenser in a folded or rolled manner;

FIG. 5A is an exemplary illustration of eye wipes of FIG. 2C that are coupled in a continuous series of eye wipes for being folded or rolled in a dispenser;

FIG. 5B is an exploded view of a perforated edge that couples consecutive eye wipes of FIG. 5A;

FIG. 6 is an exemplary roll of eye wipes of FIG. 2 that have a perforated edge between consecutive eye wipes;

FIGS. 7A and 7B are exemplary top and side views, respectively, of individual eye wipes of FIG. 2 that are adhered in a strip;

FIGS. 8A and 8B illustrate an exemplary dispenser 800 configured to store and dispense eye wipes of FIGS. 1-2;

FIG. 9 is an exemplary dispenser for dispensing eye wipes of FIGS. 1 or 2;

FIG. 10 is an exemplary dispenser having a similar structure as the dispenser of FIG. 9;

FIG. 11 is an exemplary dispenser shaped to store a roll of eye wipes as shown in FIG. 6;

FIG. 12 is another exemplary embodiment of a dispenser for storing eye wipes of FIGS. 1 or 2;

FIG. 13 is another embodiment of a dispenser for storing eye wipes of FIGS. 1 and 2;

FIG. 14 is a diagram of a conventional eye wipe 1400; and

FIG. 15 is a diagram of an eye wipe according to the principles of the present invention.

### DETAILED DESCRIPTION OF THE DRAWINGS

FIG. 1 is an illustration of an eye wipe 100, according to the principles of the present invention, sized for a user to utilize in wiping an eye. The size of the eye wipe 100 is preferably small to service the eye area in a manner preventative of accidental contact with the surface of the eye. For example, a 1″×1″, 2″×2″, or non-symmetrical dimensions may be used. For example, the eye wipe 100 may be rectangular with a combination of the exemplary dimensions. In other embodiments, the eye wipe 100 may be ovular, circular, or custom shaped for a particular application or unique design, such as in the shape of an eye.

The eye wipe 100 may be formed of a lint-free or non-lint-free material or be fibrous or non-fibrous material. The eye wipe 100 may be substantially free of chemicals or other harmful agents or irritants to an eye. For example, the eye wipe 100 may be dry or be maintained in saline solution or other “eye-friendly” solution and be substantially absent of eye-irritants, such as a non-eye irritating anti-bacterial cleanser.

FIGS. 2A-2C are illustrations of eye wipes 200a-200c (collectively 200) having different shapes and dimensions with an opening or pocket 206 for inserting a finger to control the eye wipe 200a-200c when used to wipe an eye.

Referring first to FIG. 2A, the eye wipe 200a may be composed of two portions, a first portion 202 and a second portion 204. The two portions 202 and 204 may be separate pieces connected along a front edge 205 such that a pocket 206 is formed between the two portions 202 and 204. The separate pieces forming the first portion 202 and the second portion 204 may be made of the same material or different types of material.

In one embodiment, the edge is formed at a recessed position from a front-most tip of the eye wipe 200a to avoid a seam being formed at a location that may contact an eye. In an alternative embodiment, the eye wipe 200a may be formed of a single or unitary piece of material (i) that is seamless or (ii) has a seam that is substantially hidden by a flap or other means.

FIGS. 2B and 2C depict eye wipes 200b and 200c that are shaped substantially rectangular and oval, respectively. In both cases, the pocket 206 provides at least enough volume to secure to a fingertip. Alternatively, the pocket 206 may provide at least enough volume to secure over the tip of a cotton-tipped swab. In other embodiments, the eye wipes 200 may includes straps adapted to engage a fingertip or cotton-tipped swab, grasping tabs to enable digitally grasping the wipe, or adhesive that sticks to skin thereby enabling the wipe to stick to a finger.

The texture of the material may be coarse and/or fine. For example, for removal of eye make-up, one surface of the exterior of the eye wipe 200b (i.e. wiping surface) may have a coarse texture for use in a “first pass” at eye make-up removal and another surface (i.e. wiping surface) may have a fine texture for a “final pass” at the eye make-up. Likewise, coarse and fine sides can be used to remove dried tears or wet tears, respectively, from around a baby's eyes. In one embodiment, the surfaces having different physical characteristics, for example coarse versus fine, can be on opposing sides of the exterior of the eye wipe 200.

The thickness, porosity, and other characteristics of the material composing the eye wipes 200 can be modified in various combinations. For example, the thickness at the end to be closer to the eye may be thinner than the end to be farther from the eye to support multiple uses, such as “clearing” and drying.

FIGS. 3A and 3B are illustrations of eye wipe 300a that includes an opening 306 at one end that enables the eye wipe 300a to unroll around at least a portion of a finger such that the eye wipe 300a is “form fitted” around the covered portion of the finger. In the embodiment of FIG. 3A, the rolled-up eye wipe 300a may form a disc having a thick ring 302 of material about the circumference of the disc. To assert over the finger, a user presses the finger into a center portion 304 of the eye wipe 300a and unrolls the material forming the ring 302 from the fingertip toward the knuckles until partially or fully reducing the thickness of the ring 302.

FIG. 3B is a side view of the eye wipe 300a in its extended configuration. FIG. 3C illustrates a side view of an alternative eye wipe 300b that forms a “cap” rather than a full finger cover. In both cases, the eye wipes 300 may be seamless to avoid accidental injury to an eye.

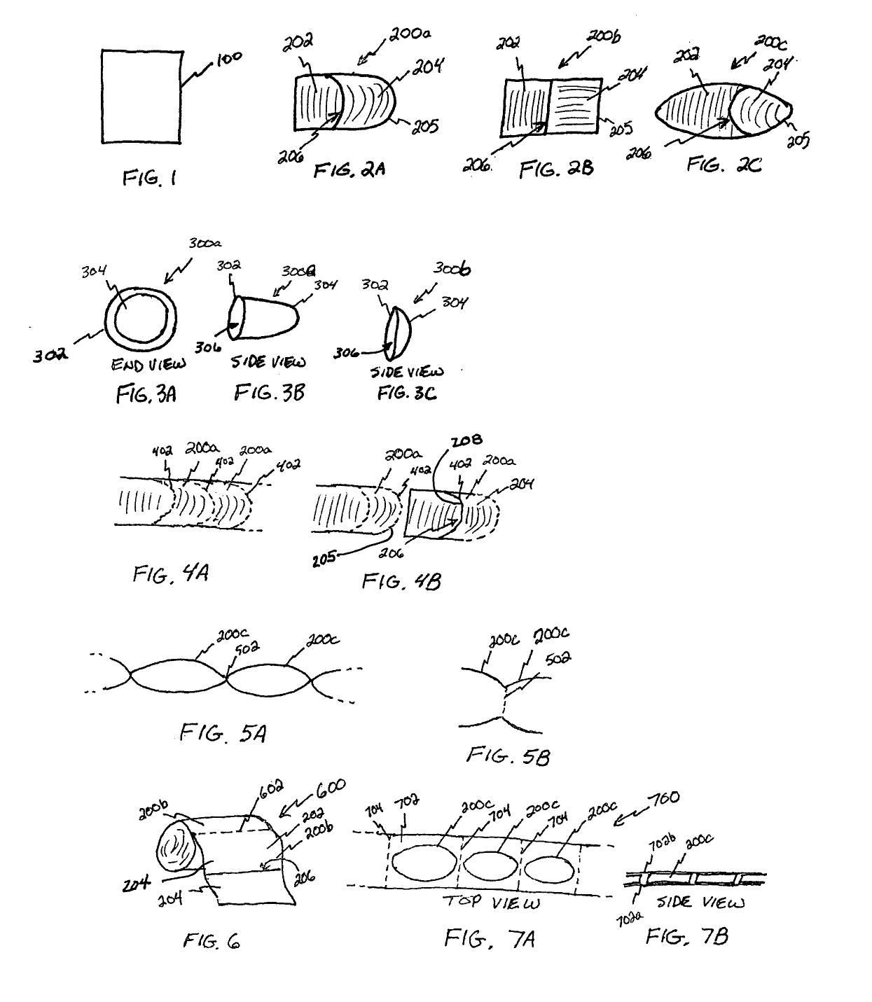

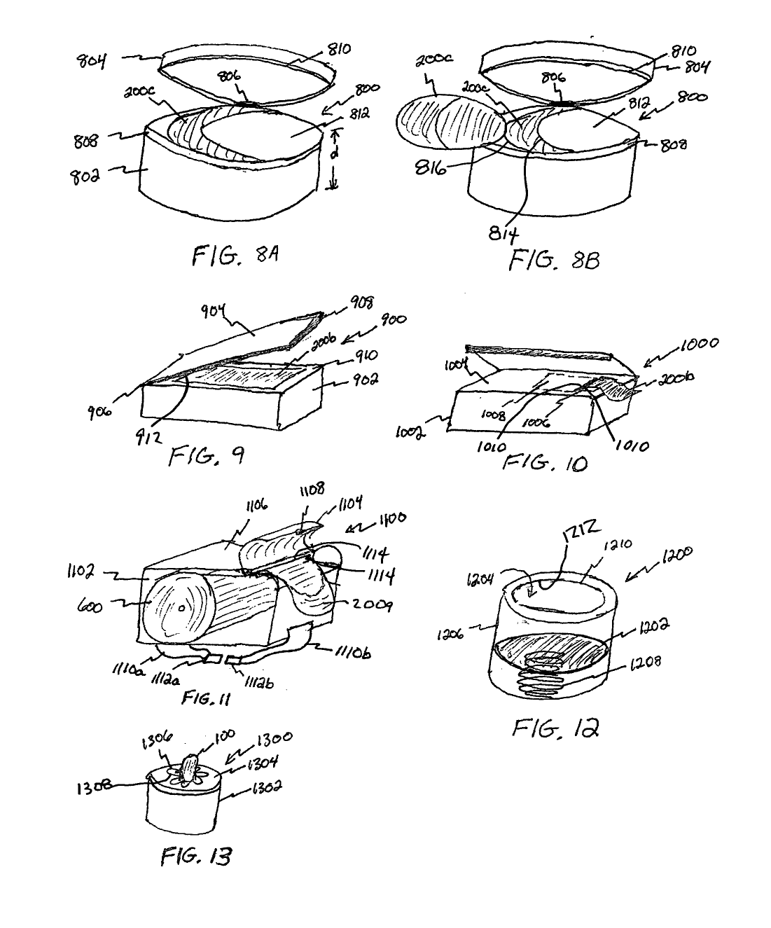

FIGS. 4A-4B illustrate eye wipes 200a that are connected together during a production or assembly process. Such an arrangement may be useful for storing the eye wipes 300 in a custom dispenser or generic package in a folded or rolled manner. The eye wipes 200a are coupled together along a perforated edge 402, which may be the front edge 205 of a first eye wipe 200a and a back edge 208 of a second eye wipe 200a. FIG. 4B illustrates the perforated edge 402 may be formed between the second portion 204 of material of one eye wipe 200a and the front edge 205 of a following eye wipe 200a in a sequence. Accordingly, the second portion 204 of material forms the pocket 206 of the eye wipe 200a when two consecutive eye wipes 200a are separated along the perforated edge 402. In another embodiment, the eye wipes 200a may include demarcations and a dispenser (see, FIG. 11) used for dispensing the eye wipes 200a may include a cutter to separate consecutive eye wipes 200a.

FIG. 5A is an illustration of eye wipes 200c of FIG. 2C that are coupled in a continuous series of eye wipes for being folded or rolled in a dispenser. The eye wipes 200c are coupled at a perforated edge 502 between consecutive eye wipes 200c.

FIG. 5B is an exploded view of the perforated edge 502 that couple consecutive eye wipes 200c. The material at a perforated edge 502 may be substantially flat or rounded such that the risk of injury to an eye, potentially caused by the resulting points of the detached perforated edge 502, is reduced. Further, the perforation may be disposed at a location on the eye wipes 200c so that the perforated edge 502 substantially avoids contact with the eye.

FIG. 6 is an exemplary roll 600 of eye wipes 200b that have a perforated edge 602 between consecutive eye wipes 200b at the side edges as referenced to the tip of the finger when worn. As shown, the eye wipes 200b are formed of the first portion 202 and second portion 204 of material that form the pocket 206. It should be understood that the eye wipes 200b may be formed from a single or multiple pieces of material. In this embodiment, the eye wipes 200b can be provided without a pocket 206 as seen in FIG. 1 and are sized for use on an eye (e.g., eye wipe 100).

FIGS. 7A and 7B are exemplary top and side views, respectively, of individual eye wipes 200c that adhere or are removably attached to a strip 700, which may be the same or different from the material composing the eye wipes 200c. As shown in FIG. 7A, the strip 700 may include one or more sheets of material 702, such as paper and/or plastic material, to which the eye wipes 200c are adhered similar to that of typical first aid devices that are used for covering a cut on skin as understood in the art. Additionally, perforations 704 may be applied between each sheet of material 702 to enable a user to separate and dispose of the sheet(s) of material 702 associated with the eye wipes 200c that are used. As shown in FIG. 7B, there are two sheets 702a and 702b (collectively 702) that are layered about the eye wipes 200c. This two-layer configuration may be utilized to maintain a higher level of sterility of the eye wipes 200c. Alternatively, a single-layer configuration to implement the strip of eye wipes 700 may be used.

FIGS. 8A and 8B illustrate an exemplary dispenser 800 configured to store and dispense eye wipes 200c. In one embodiment, the dispenser 800 is shaped in the form of an eye to store the eye wipes 200c. If the eye wipes are rectangular in shape (e.g., eye wipe 200b), the dispenser 800 may be rectangular in shape (see, for example, FIG. 9). As shown, the dispenser 800 includes a bottom member 802 having a depth (d) to accommodate storing multiple eye wipes 200c. In one embodiment, the bottom member 802 is capable of storing up to twenty-five or more eye wipes 200c. An upper member or lid 804 may be (i) coupled to the bottom member 802 along an edge 806 via a hinge (not shown), (ii) extending from the bottom member 802, or (iii) other mechanism operable to align and to raise and lower the lid 804 with respect to the bottom member 802. Alternatively, the lid 804 may be independent of the bottom member 802. However, by coupling the lid 804 with the bottom member 802, a user of the dispenser 800 is less likely to lose the lid 804 due to separation from the bottom member 802.

The bottom member 802 may include an indentation 808 or other physical feature operable to engage the lid 804. Alternatively and/or additionally, the lid 804 may include an indentation 810 or other physical feature operable to engage the bottom member 802. In one embodiment, both the bottom member 802 and lid 804 have opposing physical features that are operable to engage one another to form a seal so that the lid 804 maintains contact with the bottom member 802 unless separated with a predetermined amount of force applied by a user of the dispenser 800. In yet another embodiment, a snap, clasp, clip, ridge, or other securing member may be integrated into either or both the lid 804 and/or bottom member 802.

In another embodiment, the dispenser 800 may be utilized to maintain moisture for the eye wipes 200c in the case of producing the eye wipes 200c with an eye friendly solution, e.g., saline solution, and that the seal created by the bottom member 802 and lid 804 provides the moisture maintaining functionality, at least in part. A sealing ring (not shown) may be employed if the eye wipes 200c are to be stored in a liquid solution or if a separate compartment is included with the dispenser 800 containing a liquid solution.

Further shown in FIGS. 8A and 8B is a flap 812 that may be pliable. The flap 810 may be utilized to press down on the eye wipes 200c to apply pressure to the eye wipes 200c to allow a user to extract a single (i.e., top) eye wipe 200c from the dispenser 800. In the case of the eye wipes 200c being coupled by a perforated edge 502 (FIG. 5), pressing on the flap 840 allows the user to tear along the perforated edge 502 so as to separate the eye wipes 200c. To maintain the eye wipes 200c at the top of the bottom member 802, a spring loaded or compressed member (not shown) may be included below the eye wipes 200c. Furthermore, the flap 812 may be provided with a flexible seal member 814 on its leading edge that cooperates with a flexible seal member 816 on the bottom member 802 to at least partially seal moisture into the bottom portion 802 and substantially maintain sterility of the eye wipes 200a remaining in the bottom portion 802 as eye wipes 200a are dispensed.

FIG. 9 is an exemplary dispenser 900 for dispensing eye wipes 100 or 200 (FIGS. 1 and 2). The dispenser 900 may be rectangular in shape and have a bottom member 902 and a lid 904 coupled thereto via an edge 906. The edge 906 operable to couple the lid 904 and bottom member 902 may be any of the edges of the bottom member 902. Similar to the dispenser 800 of FIG. 8, the lid 904 may be adapted to seal in moisture within the dispenser to maintain moist eye wipes 200b by utilizing a compressible material, such as rubber or other pliable material, along the edges 908 of the lid 904 or along a top rim 910 of the bottom member 902. Additional pliable seals 912 may be provided where the eye wipes 200b exit the bottom member 902 to cooperate in at least partially sealing against escape of moisture and substantially maintaining sterility of the eye wipes 200b remaining in the bottom member 902. It should be understood that the bottom member 902 and lid 904 may be shaped to accommodate different shaped eye wipes 200. Because the eye wipes 200b are sized to engage a finger, the dispenser may accommodate the size of the eye wipes 200b, thereby having a maximum length or major axis of about two-inches or less.

FIG. 10 is an exemplary dispenser 1000 having a similar structure as the dispenser 900 of FIG. 9. However, to better provide for maintaining moisture within the dispenser 1000, a bottom member 1002 may include a top surface 1004 that covers a substantial portion of the area of the bottom member 1002. A slit 1006 may be disposed on the top surface 1004 to enable the eye wipes 200b to be dispensed therethrough. The slit 1006 may include opposing pliable seals 1010 to at least partially seal against escape of moisture and substantially maintain sterility of the eye wipes 200b remaining in the bottom member 1002. In one embodiment, a flap 1008 may be disposed on the top surface 1004 to enable the user to press down on eye wipes 200b not desired to be removed from the dispenser 1000. Utilizing eye wipes 200b that are coupled via a perforated edge may enable easier dispensing characteristics. Alternatively, if eye wipes 200b are formed as a continuous roll or folded stack and not perforated, a tooth shaped member (not shown) as understood in the art may be integrated with the dispenser 1000 or coupled thereto to enable a user to separate consecutive eye wipes 200b.

FIG. 11 is an exemplary dispenser 1100 that is shaped to store a roll 600 of eye wipes 200a as shown in FIG. 6. The roll 600 may be stored in a main compartment 1102 and separate compartments (not shown) may be utilized to allow storage of other items, such as items used for infants. A lid 1104 may be coupled to a top member 1106 of the dispenser 1100 to enable the lid 1104 to raise and lower to allow a user to access and store the eye wipes 200a conveniently. Accordingly, the lid 1104 may maintain moisture, if utilized, for the eye wipes 200a. A snap 1108 or other securing member may be utilized to maintain the lid 1104 with another member of the dispenser 1100. Furthermore, pliable seals 1114 may be provided where the eye wipes 200a exit the main compartment 1102 to cooperate in at least partially sealing against escape of moisture and substantially maintaining sterility of the eye wipes 200a remaining in the main compartment 1102.

The dispenser 1100 may further include one or more members for securing the dispenser 1100 to a flexible or stiff item or object. A flexible item may include a diaper bag, purse strap, back pack, carry bag, etc. A stiff item may include a carriage handle, crib frame, car dashboard, and so forth. As shown, straps 1110a and 1110b (collectively 1110) may be coupled to the dispenser 1100 and have clips 1112a and 1112b (collectively 1112) attached thereto, respectively. The straps 1110 may be wrapped around a stiff item, for example, and the clips 1112 may secure the dispenser 1100 thereto. It should be understood that other securing members adapted to secure the dispenser 1100 to a flexible or stiff item may alternatively be utilized. Still yet, other securing mechanisms may be utilized, such as a magnet or minute hook fastening device.

FIG. 12 is another exemplary embodiment of a dispenser 1200 according to the principles of the present invention. As shown, the dispenser 1200 may include a platform 1202 that may slide or translate within in a cavity 1204, which is formed by a sidewall 1206 of the dispenser 1200. A spring 1208 or other force providing mechanism may be utilized to translate the platform 1202 within the dispenser 1200. Eye wipes (not shown) may be stored between the platform 1202 and opening 1210 of the cavity 1204 of the dispenser 1200 so that the eye wipes are continuously being pushed to the opening 1210 so as to be readily available to the user. The opening 1210 can include a pliable seal member 1212 about its perimeter to at least partially seal against escaping moisture and substantially maintain sterility of the wipes remaining in the dispenser 1200.

FIG. 13 is another embodiment of a dispenser 1300 for storing eye wipes 100 and 200 of FIGS. 1 and 2. The dispenser 1300 may include a bottom member 1302 that stores the eye wipes 100 and 200 and lid 1304 having at least one opening 1306 operable to maintain an eye wipe 100 and 200 extending therethrough, thereby being readily available for the user to remove from the dispenser 1300. The at least one opening 1306 can include a pliable seal member 1308 about its perimeter to at least partially seal against escaping moisture and substantially maintain sterility of the wipes remaining in the dispenser 1300.

While the above-discussed dispensers provide for multi-eye wipe storage and dispensing, the eye wipes 100 and 200 may also be individually wrapped in hermetically or non-hermetically sealed packages to provide a convenient form for the user. The individually wrapped eye wipes may be contained in a dispenser similar to those discussed above or in a conventional box as understood in the art.

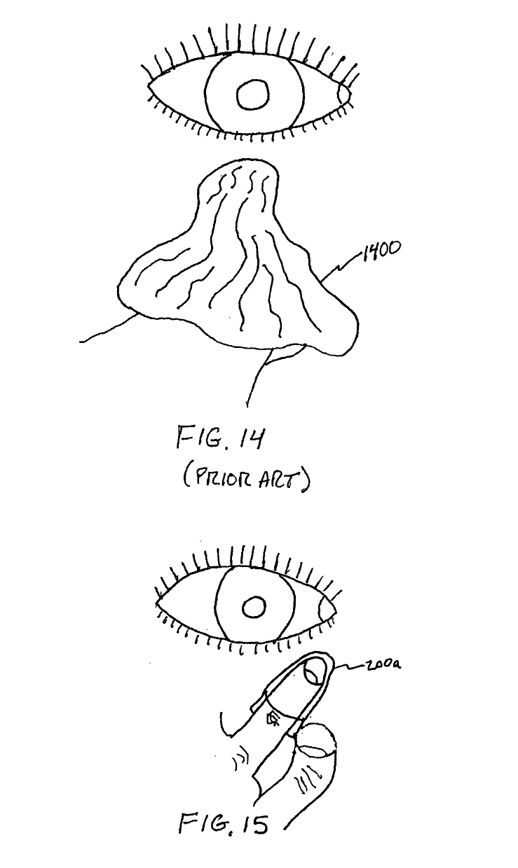

FIG. 14 is a diagram of a conventional eye wipe 1400. The conventional eye wipe 1400 may be a tissue other material having a similar size (e.g., 6-by-8 inches). The conventional eye wipe 1400 may be infused with eye irritating substances or solutions, for example, dyes, chemicals, perfumes, soaps, alcohol, lotions, or other eye irritant substances.

FIG. 15 is a diagram of an eye wipe according to the principles of the present invention. In one embodiment, the eye wipe 200a is sized to fit one or two fingers of an adult or child. The eye wipe 200a, being sized for a finger, provides for control and is environmentally friendly. The eye wipe 200a may provide for absorption of tears and discharge, wet to gently clear debris, and include a solution that does not irritate or cause allergic reaction to an eye. Herbal moistening or cleansing ingredients may be utilized, where the herbal ingredients may include eyebright herb, fennel, goldenseal, chamomile, raspberry leaf, blue vervain or bayberry, or a combination of two or more herbal or non-herbal ingredients. Alternatively, the eye wipe 200a may include or be saturated with a solvent that enables removal of eye makeup.

The previous description is of at least one embodiment for implementing the principles of the present invention, and the scope of the invention should not necessarily be limited by this description. The scope of the present invention is instead defined by the following claims.

What is claimed is:

1. An eye wipe, comprising:
an eye wiping surface;
a second surface opposite said eye wiping surface; and
a member on the second surface configured to engage a finger.

2. The eye wipe according to claim 1, wherein said member is coupled to said second surface to form an elongate cavity.

3. The eye wipe according to claim 2, wherein the cavity is substantially tubular having an opening at one end and tapering at the other end.

4. The eye wipe according to claim 1, wherein said eye wiping surface is substantially absent of fibrous elements.

5. The eye wipe according to claim 1, wherein said eye wiping surface, second surface, and member are composed of a unitary piece of material.

6. The eye wipe according to claim 1, wherein said eye wiping surface is operable to absorb moisture.

7. The eye wipe according to claim 1, wherein said eye wiping surface has at least one of the following shapes: square, rectangular, oval, and circular.

8. The eye wipe according to claim 1, wherein said eye wiping surface has a maximum dimension of less than about two inches.

9. The eye wipe according to claim 1, wherein said eye wiping surface is substantially absent of an eye irritant solution.

10. The eye wipe according to claim 1, wherein the eye wiping surface includes a non-eye irritating solution.

11. The eye wipe according to claim 1, wherein the eye wiping surface includes a solution operable to remove makeup.

12. An wipe, comprising:
a member having a first surface and a second surface, the first surface operable to absorb liquid, said member forming a tubular cavity with an open end and a closed end.

13. The wipe according to claim 12, wherein said member is operable to be configured in a state forming essentially a disc.

14. The wipe according to claim 12, wherein the open end is substantially circular.

15. The wipe according to claim 12, wherein the tubular cavity is sized to receive a finger.

16. The wipe according to claim 12, wherein said member has a maximum dimension of less than about two inches.

17. A method for dispensing eye wipes, said method comprising:
forming a series of eye wipes; and
storing the series of eye wipes in a dispenser in a manner facilitating separation of an eye wipe from the series of eye wipes.

18. The method according to claim 17, wherein said forming includes applying a perforation between two eye wipes in the series of eye wipes.

19. A dispenser operable to store and dispense eye wipes, said dispenser comprising:
a bottom member configured to store a plurality of eye wipes and dispense a single eye wipe at a time; and
a lid operable to engage said bottom member.

20. The dispenser of claim 19, wherein said bottom member has a maximum length or major axis of about two-inches.

21. The dispenser of claim 19, wherein said bottom member and lid are operable to seal liquid when coupled.

22. The dispenser of claim 21, further comprising a mechanism coupled to an exterior surface of either said bottom member or lid and operable to be secured to another object.
